(19) United States
(12) Patent Application Publication
(10) Pub. No.: US 2018/0318611 A1
(43) Pub. Date: Nov. 8, 2018

(54) DEVICE FOR ACOUSTIC COUPLING, TEMPERATURE REGULATION, AND IMMOBILIZATION OF PATIENT DURING FOCUSED ULTRASOUND THERAPY

(71) Applicants: Hong Chen, St. Louis, MO (US); Michael B. Altman, St. Louis, MO (US); Satya V.V.N. Kothapalli, St. Louis, MO (US)

(72) Inventors: Hong Chen, St. Louis, MO (US); Michael B. Altman, St. Louis, MO (US); Satya V.V.N. Kothapalli, St. Louis, MO (US)

(73) Assignee: Washington University, St. Louis, MO (US)

(21) Appl. No.: 15/974,165

(22) Filed: May 8, 2018

Related U.S. Application Data
(60) Provisional application No. 62/503,029, filed on May 8, 2017.

(51) Int. Cl. A61N 7/02

(52) U.S. Cl. CPC A61N 7/02

(57) ABSTRACT

A device to acoustically couple a patient to an MR-HIFU system includes a first membrane configured to acoustically couple to an ultrasound window of the MR-HIFU system, a second membrane configured to acoustically couple to the patient at an exposed region overlying an ultrasound focus of the MR-HIFU system, a continuous side wall that includes a first edge sealed to the first membrane and a second edge sealed to the second membrane, and an acoustic coupling fluid contained within a closed internal volume defined by the first membrane, the second membrane, and the continuous side wall. The device also includes a heat exchange device operatively coupled to the acoustic coupling fluid. The device is configured to form an impedance-matched ultrasound coupling between the ultrasound window and the exposed region of the patient and is further configured to regulate a temperature of the exposed region of the patient.

### CROSS-REFERENCE TO RELATED APPLICATION

This application claims the benefit of U.S. Provisional Application Ser. No. 62/503,029, filed May 8, 2017, entitled DEVICE FOR ACOUSTIC COUPLING, TEMPERATURE REGULATION, AND IMMOBILIZATION OF PATIENT DURING FOCUSED ULTRASOUND THERAPY, which is hereby incorporated in its entirety herein.

### BACKGROUND

Ultrasound is used as an imaging modality to enable a variety of versatile medical visualization tools. Further, ultrasound is increasingly used for a wide variety of therapeutic interventions that enable non-invasive treatment of deep tissues with minimal effect on overlying organs and tissues. Therapeutic interventions incorporating targeted delivery of high-intensity focused ultrasound (HIFU) that are currently in use or under development include thermal ablation of benign growths such as fibroids within the uterus, breast, and other tissues, as well as cancerous lesions in a variety of locations, such liver, brain, breast, prostate, and lung tissues. Other treatments make use of focused ultrasound (FUS) for the targeted ablation of brain tissues for the treatment of brain disorders including essential tremors, neuropathic pain, and Parkinsonian tremors. Mild hyperthermia (HT) is another therapeutic technique in which tissue is heated, using FUS delivered at a lower energy, to temperatures elevated above body temperature, but below ablative temperatures. FUS is also under development for other therapies based on its mechanical bioeffects, such as focused ultrasound-induced blood-brain barrier opening, boiling histotripsy for tissue emulsification, neuromodulation with pulsed focused ultrasound, and thrombolysis using ultrasound with microbubbles.

HIFU therapy, currently in clinical use, is typically administered by insonifying a tissue of interest with high intensity ultrasound that is absorbed by the cells within a targeted area and converted into heat, thereby raising the temperature of the respective tissues. Depending on the intensity of the ultrasound energy delivered to the tissues, the temperature may rise to mild hyperthermic levels ranging from about 38° C. to about 45° C. or to higher ablation temperatures in excess of about 55° C. In the case of ablation, as the local tissue temperature rises above about 55° C., coagulative necrosis of the tissues occurs, resulting in immediate cell death.

In general, the transducers used to produce and deliver the HIFU treatment may be positioned outside the body or may be inserted into the body e.g. through blood vessels, urethra, rectum etc. In some devices, a HIFU device may be coupled with an MRI scanner to enable MR-guided HIFU (MR-HIFU) that enables the targeting of the delivered HIFU to a relatively precise region within the deep tissues of a patient, as well as thermal monitoring of the deep tissues using MR thermal measurement methods.

FIG. 1 is a schematic illustration of a representative existing MR-HIFU system 10. Although the MR-HIFU system 10 enables relatively precise targeting of the delivery of ultrasound energy to deep tissues, the design of existing MR-HIFU systems 10 must compromise between efficient focusing and delivery of the HIFU and sufficiently high resolution MR imaging of the targeted deep tissue region of the patient. At least some existing MR-HIFU systems 10 include a focused ultrasound transducer or transducer array 12 positioned within an oil bath 14 positioned below the surface of a table 16 upon which MR imaging (not shown) is also performed on a patient 18, as illustrated in FIG. 1. In this design, the HIFU 20 is delivered to a targeted deep tissue region 22 through an ultrasound window 24 formed within the table surface 26. Typically, the exposed surface 28 of the ultrasound window is a planar surface or another fixed contour selected to enhance the acoustic coupling of the transducer in the oil bath 14 with an exposed surface 30 of the patient 18 overlying the targeted deep tissue region 22. In some instances, there may exist mismatches between the respective contours of the ultrasound window 24 of the MR-HIFU system and the exposed surface 30 of the patient 18 overlying the targeted deep tissue region 22 that may reduce the efficiency of transmission of the HIFU 20 into the exposed surface 30 of the patient 18. In addition, the fixed contour of the ultrasound window 24 of typical existing MR-HIFU systems 10 may limit the targeted deep tissue regions 22 that may be treated. For example, a planar ultrasound window 24 may be relatively well-suited for MR-HIFU treatment of uterine fibroids, but not for the treatment of brain due to the relatively high curvature of the skull containing the targeted deep tissue region within the brain.

In certain instances, one or more gel packs 32 may be inserted between the exposed surface 30 of the patient 18 and the ultrasound window 24 to compensate for these mismatches between the contours of the ultrasound window 24 of the MR-HIFU system 10 and the patient 18. However, the insertion of the gel packs 32 between the patient 18 and the ultrasound window 24 may introduce additional issues that may reduce the effectiveness of the MR-HIFU system 10 and/or patient comfort during treatment. For example, the gel packs may increase the potential for patient movement during treatment, thereby reducing the accuracy of the delivered HIFU. In addition, the gel packs may thermally isolate the surface of the patient from the surface of the ultrasound window, thereby reducing the MR-HIFU system's ability to regulate the temperature of the patient's skin and other tissues overlying the targeted deep tissue region of treatment. For example, some existing MR-HIFU systems may provide active cooling to the ultrasound window to provide cooling to the patient's skin, but the insertion of a gel pack between the patient and the ultrasound window significantly reduces the effectiveness of this active cooling.

### SUMMARY

In one aspect, a device to acoustically couple a patient to an MR-HIFU system is disclosed. The device includes a first membrane configured to acoustically couple to an ultrasound window of the MR-HIFU system, a second membrane configured to acoustically couple to the patient at an exposed region overlying an ultrasound focus of the MR-HIFU system, a continuous side wall that includes a first edge sealed to the first membrane and a second edge sealed to the second membrane, an acoustic coupling fluid contained within a closed internal volume defined by the first membrane, the second membrane, and the continuous side wall, and a heat exchange device operatively coupled to the acoustic coupling fluid. The device is configured to form an impedance-matched ultrasound coupling between the ultrasound window and the exposed region of the patient and is further configured to transfer heat between the exposed region of the patient and the acoustic coupling fluid via the second membrane.

In another aspect, a method of acoustically coupling and thermally modulating a patient during treatment using an MR-HIFU system is disclosed. The method includes providing a device that includes a first membrane configured to acoustically couple to an ultrasound window of the MR-HIFU system, a second membrane configured to acoustically couple to the patient at an exposed region overlying an ultrasound focus of the MR-HIFU system, a continuous side wall that includes a first edge sealed to the first membrane and a second edge sealed to the second membrane, an acoustic coupling fluid contained within a closed internal volume defined by the first membrane, the second membrane, and the continuous side wall, and a heat exchange device operatively coupled to the acoustic coupling fluid. The method also includes positioning the first membrane of the device adjacent to at least a portion of the ultrasound window of the MR-HIFU system, positioning the exposed region of the patient adjacent to the second membrane, and modulating a temperature of the exposed region by transferring heat between the exposed region of the patient and the acoustic coupling fluid as well as modulating a fluid temperature of the acoustic coupling fluid using the heat exchanger.

### BRIEF DESCRIPTION OF THE FIGURES

The following figures illustrate various aspects of the disclosure.

FIG. 1 is a cross-sectional schematic illustration of an existing MR-HIFU system;

FIG. 2 is a cross-sectional side view of a device according to one aspect of the disclosure;

FIG. 3 is a cross-sectional top view of a device according to one aspect of the disclosure;

FIG. 4 is a cross-sectional side view of a device according to one aspect of the disclosure shown acoustically coupled to the MR-HIFU system and patient illustrated in FIG. 1;

FIG. 5 is a cross-sectional side view of a device according to one aspect of the disclosure that is provided with an inlet and exit port for recirculation of acoustic coupling fluid;

FIG. 6 is a top view of a reinforced second membrane according to one aspect of the disclosure; and

FIG. 7 is a cross-sectional side view of a device according to one aspect of the disclosure that is provided with the reinforced second membrane of FIG. 6.

While multiple embodiments are disclosed, still other embodiments of the present disclosure will become apparent to those skilled in the art from the following detailed description, which shows and describes illustrative embodiments of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

In various aspects, a device is disclosed for acoustically coupling a patient to an MR-HIFU system and to exchange heat between an exposed region of the patient that is acoustically coupled to the device and an acoustic coupling fluid contained within the device. The temperature of the acoustic coupling fluid is modulated by a heat exchange device operatively coupled to the acoustic coupling fluid. In addition, the device may be provided with additional elements, described in detail below, that enable reversibly securing the device to an ultrasound window of an MR-HIFU system, and that enable reversibly immobilizing the patient relative to the device.

In various aspects, the device enables at least several functions that enhance the effectiveness of a clinical MR-HIFU treatment. The device provides efficient acoustic coupling for transmitting HIFU into the targeted deep tissue regions of the patients. The device further provides for temperature regulation by heat exchange at the exposed surface of the patient in contact with the device. By way of non-limiting example, skin burn is a commonly reported side effect during HIFU treatment, and the thermal modulation capability enabled by the device as described herein below reduces the likelihood of skin burn through active cooling by the device. By way of another non-limiting example, this thermal regulation capability may facilitate particular applications of MR-HIFU treatments including, but not limited to, mild hyperthermia (HT), where it may be desired to hold the skin at body temperature or at a warmer level.

In some aspects, the device further enables immobilization of the patient, thereby ameliorating a significant technical challenge in MR-HIFU treatment. Without being limited to any particular theory, unintentional patient motion may lead to misalignment of a HIFU treatment and/or may lead to errors in the MR temperature monitoring. Patient immobilization elements of the device configured to prevent accidental motion of the treatment target are described in additional detail below. In addition, the device enables the ability to conform the interface contours of the device to the variable anatomy of individual patients and MR-HIFU systems while enabling all of the additional features of the device described herein.

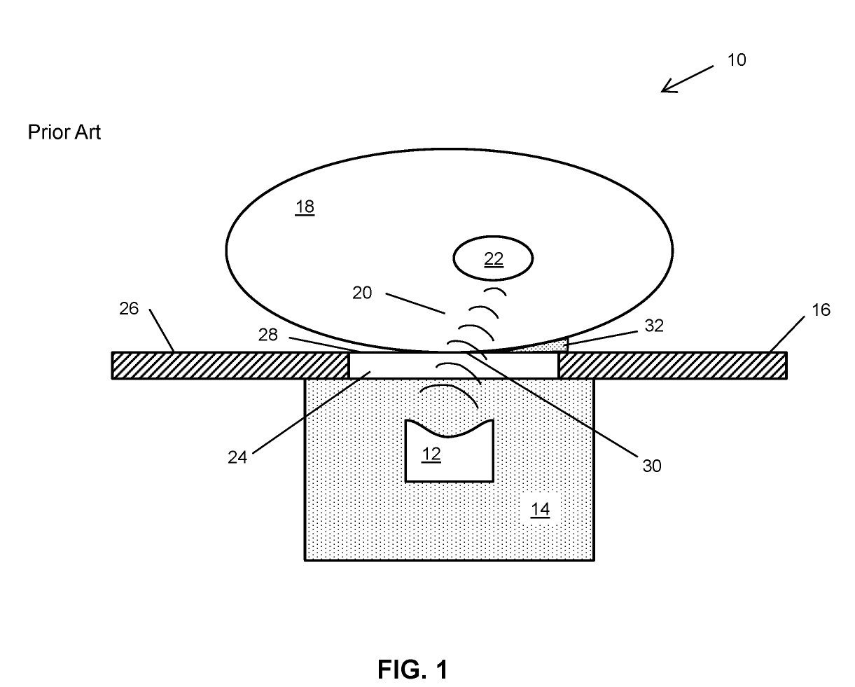

In various aspects, the device overcomes at least several shortcomings of existing MR-HIFU systems and associated devices such as gel bags. In various aspects, the device simultaneously provides enhanced acoustic coupling and temperature regulation for the patient. Some existing systems, such as the MR-HIFU system 10 illustrated in FIG. 1 may provide for thermal regulation via heat exchange through the ultrasound window 24, but this capability is diminished if additional elements, such as gel packs 32, are inserted between the patient 18 and the ultrasound window 24 to achieve sufficient acoustic coupling to transmit the HIFU 20 to the targeted deep tissue region 22 with sufficient energy to exert the desired effect on cells within the targeted deep tissue region 22.

In various other aspects, the device includes adjustable ultrasound window interface contours and patient interface contours configured to provide flexibility with respect to treatments that may be conducted for a variety of different MR-HIFU systems. At least some existing MR-HIFU systems are provided with contoured interface surfaces selected to enhance the effectiveness of treatment of a particular region of a patient that renders the MR-HIFU system less effective at treating other regions that do not share the same interface contour. The flexible interface contours provided by the device in various aspects enables a single MR-HIFU system to be adapted to perform treatments on a wider variety of patient regions than was previously achievable.

In other aspects, the device provides the capability to immobilize the patient relative to the MR-HIFU system, while simultaneously providing enhanced acoustic coupling and thermal management to the patient as well. At least some existing systems provide for patient immobilization, but the immobilization may be compromised by the introduction of additional elements such as gel bags to enhance acoustic coupling of the patient to the MR-HIFU system.

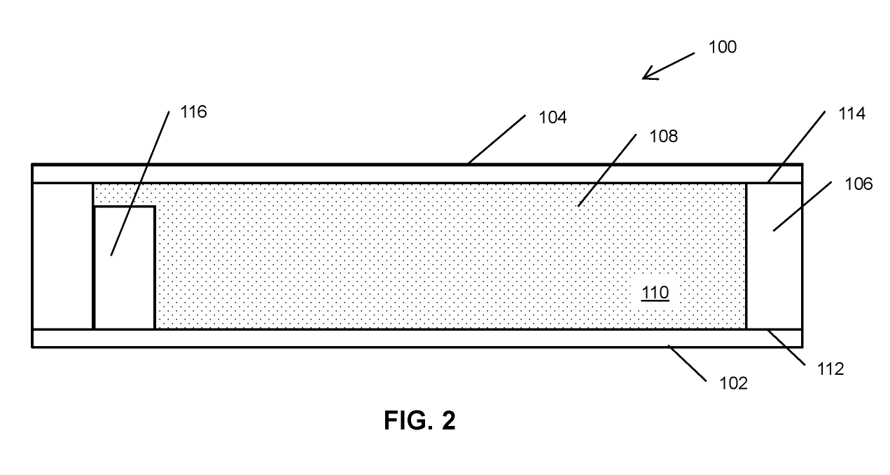

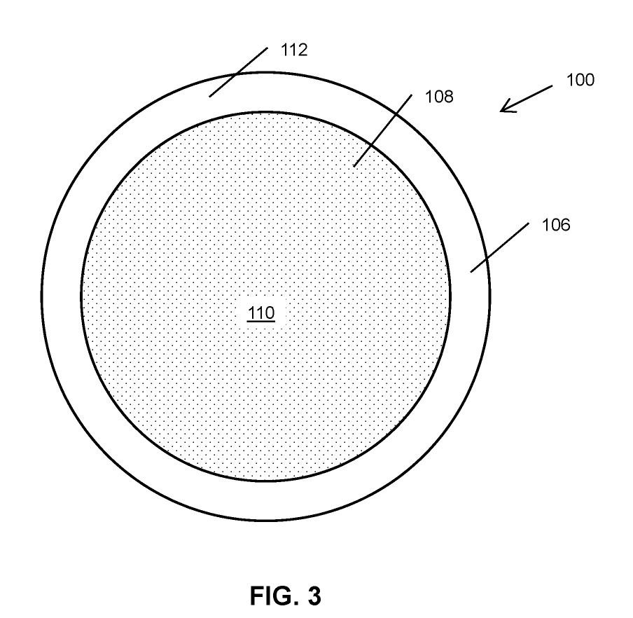

FIG. 2 and FIG. 3 are cross-sectional side and top views, respectively, of the device 100 in one aspect. In this aspect, the device 100 includes a first membrane 102, a second membrane 104, and a continuous side wall 106 that together define a closed internal volume 108 containing an acoustic coupling fluid 110. The first membrane 102 is sealed to the continuous side wall 106 at a first edge 112 of the side wall 106, and the second membrane 104 is sealed to the continuous side wall 106 at a second edge 114 of the side wall 106 to enclose the closed internal volume 108.

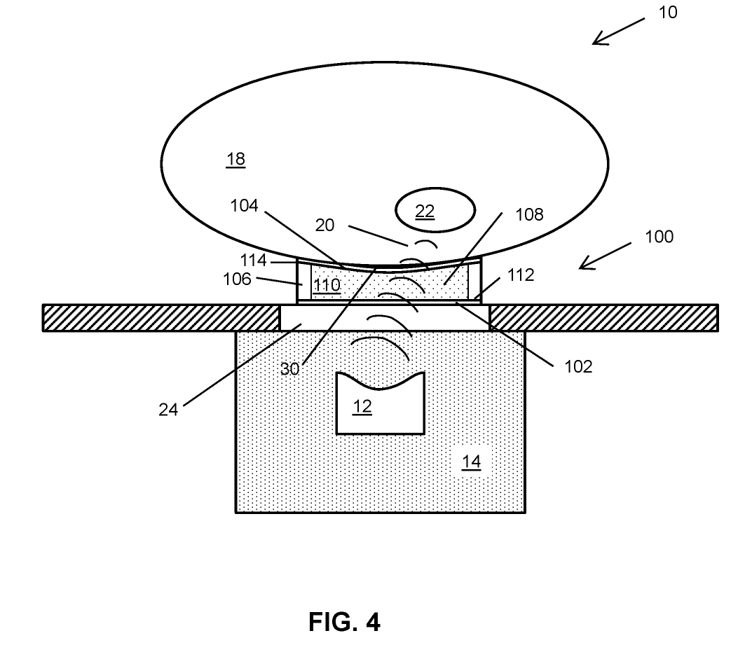

FIG. 4 is a cross-sectional schematic view of the device 100 shown acoustically coupled to the MR-HIFU system 10 and to the patient 18 in one aspect. In various aspects, the first membrane 102 is configured to acoustically couple with the ultrasound window 24 and the second membrane 104 is configured to acoustically couple with the exposed surface 30 of the patient 18 that overlies the targeted deep tissue region 22. In an aspect, the first edge 112 and the second edge 114 of the side wall 106 may further define first and second contours, respectively that are configured to conform to the contours of the ultrasound window 24 and the exposed surface 30 of the patient 18, respectively. In this aspect, the first and second contours enable more intimate contact of the interfacing surfaces, thereby enhancing the acoustic coupling at the device-ultrasound window interface and at the device-patient interface. In various aspects, a layer of an additional acoustic coupling material including, but not limited to, an ultrasound coupling gel, may be inserted between the first membrane 102 and the ultrasound window 24 and between the second membrane 104 and exposed surface 30 of the patient 18 in order to enhance the acoustic coupling at the two interfaces.

As illustrated in FIG. 4, the device 100 in various aspects provides acoustic coupling of the patient 18 to the ultrasound window 24 of the MR-HIFU system 10. HIFU 20 produced by the focused ultrasound transducer or transducer array 12 in the oil bath 14 of the MR-HIFU system 10 propagates through the ultrasound window 24 and across the first membrane 102, across the acoustic coupling fluid 110, across the second membrane 104 and into the body of the patient 18. As described below, the dimensions and materials of the various elements of the device 100 are selected to reduce mismatches in acoustic impedance across each of the interfaces at which different materials abut. Without being limited to any particular theory, losses in acoustic energy of the HIFU are reduced during propagation from the transducer 12 to the targeted deep tissue region 22 by matching the acoustic impedance of materials of the device as practicable.

Referring again to FIG. 4, the device 100 may further enable thermal management of the tissues of the patient 18 overlying the targeted deep tissue region 22 in various aspects. In an aspect, the acoustic coupling fluid 110 may act as a heat source or heat sink as needed to modulate the temperature of the tissues of the patient 18. In one aspect, the second membrane is configured to enable heat exchange between the tissues of the patient 18 and the acoustic coupling fluid 110 via the exposed surface 30 of the patient 18.

In various aspects, the device 100 may further include a heat exchange device 116 that is operatively coupled to the acoustic coupling fluid 110. In these various aspects, the heat exchange device 116 is configured to heat or cool the acoustic coupling fluid 110 as needed to modulate the tissue temperatures of the patient 18 via heat exchange between the acoustic coupling fluid 110 and the exposed surface 30 of the patient. In one aspect, illustrated in FIG. 2, the heat exchange device 116 may be a heat exchanger positioned within the closed internal volume 108 within the device 100. In this aspect, the acoustic coupling fluid 110 remains relatively stationary within the closed internal volume 108.

Any known suitable heat exchange device 116 may be selected for use as a heat exchanger in the device. Non-limiting examples of suitable heat exchange devices include Peltier heat pumps, water chillers, resistive heaters, and any other suitable heat exchange device.

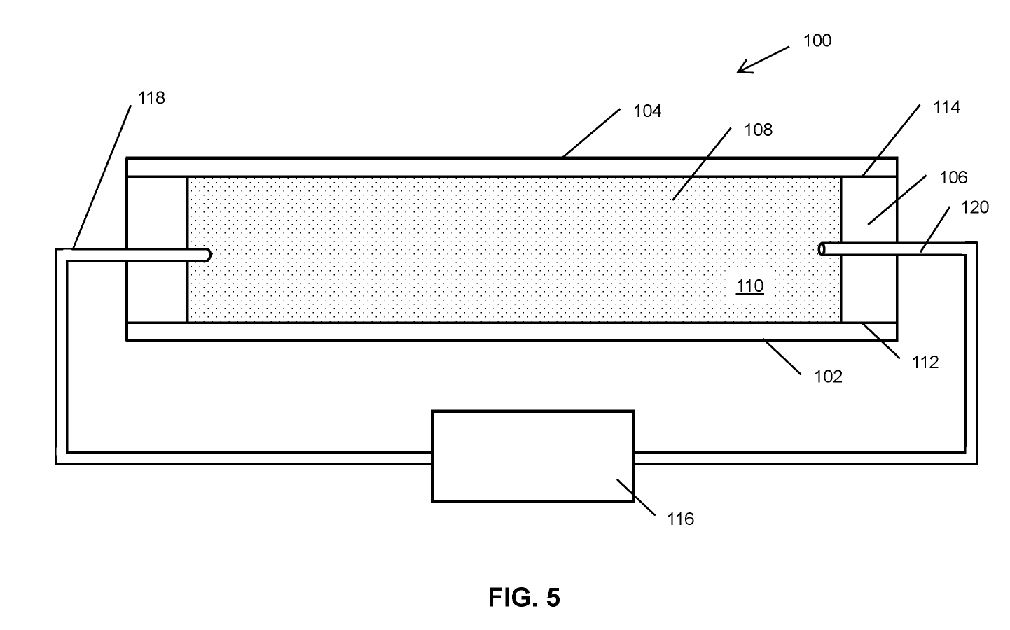

In various other aspects, the heat exchange device may be a recirculating heat exchange device 116 that makes use of a circulating coolant. FIG. 5 is a cross-sectional side view of a device 100 that provides for the inclusion of a recirculating heat exchange device 116. As illustrated in FIG. 5, the side wall 106 defines an inlet port 118 and an exit port 120. The exit port 120 is configured to remove a portion of the acoustic coupling fluid 110 out of the closed internal volume 108, and the inlet port 118 is configured to return the portion of the acoustic coupling fluid 110 removed via the exit port 120 back into the closed internal volume 108 in a constant recirculation pattern. In this aspect, the recirculating heat exchange device 116 exchanges heat with the portion of the acoustic coupling fluid 110, which acts as the recirculating coolant, received from the exit port 120. The recirculating heat exchange device 116 further returns the portion of acoustic coupling fluid 110, which has been heated or cooled by the recirculating heat exchange device 116 back to the closed internal volume 108 via the inlet port 118. Any suitable known recirculating heat exchange device may be selected for incorporation into the device without limitation.

In various additional aspects, the device 100 may further make use of thermal management features included in the MR-HIFU system 10. By way of non-limiting example, some existing MR-HIFU systems 10 are provided with thermal regulation capability via a temperature-controlled ultrasound window 24. Referring again to FIG. 4, if the ultrasound window 24 is actively heated or cooled, the device 100 may further enable heat exchange between the ultrasound window 24 and the acoustic coupling fluid 110 via the first membrane 102. In this aspect, the added thermal regulation capability enhances the available heat exchange capacity with respect to the acoustic coupling fluid.

In various aspects, the device may further include one or more sensors including, but not limited to one or more thermometers to monitor temperatures of the patient's skin, the acoustic coupling fluid, and any other relevant temperature. In an aspect, the monitored temperatures may be used as feedback to a control system configured to operate the heat exchange elements of the device. In an additional aspect, the sensor may be a cavitation detector to monitor any bubbles trapped inside the acoustic coupling fluid or the interface between the device and the patient's skin.

As described above, the first membrane 102 and second membrane 104 are configured to efficiently propagate HIFU into the patient and to efficiently exchange heat between the patient and the acoustic coupling fluid. In one aspect, the materials for the construction of the first and second membranes may be selected to be thermally and acoustically transmissive. Without being limited to any particular theory, it is thought that a reduced thickness of the first and second membranes enhances the efficiency of both heat and acoustic transmission. With respect to acoustic transmission, it is thought that a membrane thickness that is no more than about ½ of the acoustic wavelength may enhance the efficiency of acoustic transmission. By way of non-limiting example, assuming an ultrasound frequency of about 1 MHz, the wavelength of ultrasound in water and tissues is about 1.5 mm. In one aspect, the first and second membranes are less than about 1 mm in thickness, depending on the acoustic wavelength.

Any suitable thermally and acoustically transmissive material may be selected for the construction of the first and second membranes without limitation. In one aspect, the first and second membranes may be constructed from a vylidene chloride material including, but not limited to polyvylidene chloride. By way of non-limiting example, the first membrane 102 may be constructed from a plastic food wrap film including, but not limited to, STRETCH-TITE® plastic food wrap (Polyvinyl Films, Inc., Sutton. Mass., USA). BY way of another non-limiting example, the second membrane 104 may be constructed from an ultrathin silicone film (ELASTOSIL®, Wacker Chemie AG, München, Germany).

In various aspects, the contact area of the first and second membranes may be any suitable size for enabling MR-HIFU therapy of a patient without limitation. In one aspect, the contact area of the first membrane may be selected so that the first membrane covers at least a portion of the ultrasound window of the MR-HIFU device. In another aspect, the contact areas of the first and second membranes may be selected to accommodate a range of angular HIFU trajectories obtainable from the transducer of the MR-HIFU device to the exposed region of the patient. In various aspects, the respective contact areas of the first and second membranes may be selected independently. Consequently, the contact area of the first and second membranes may be essentially equal, the contact area of the first membrane may be larger than the contact area of the second membrane, or the contact area of the first membrane may be smaller than the contact area of the second membrane.

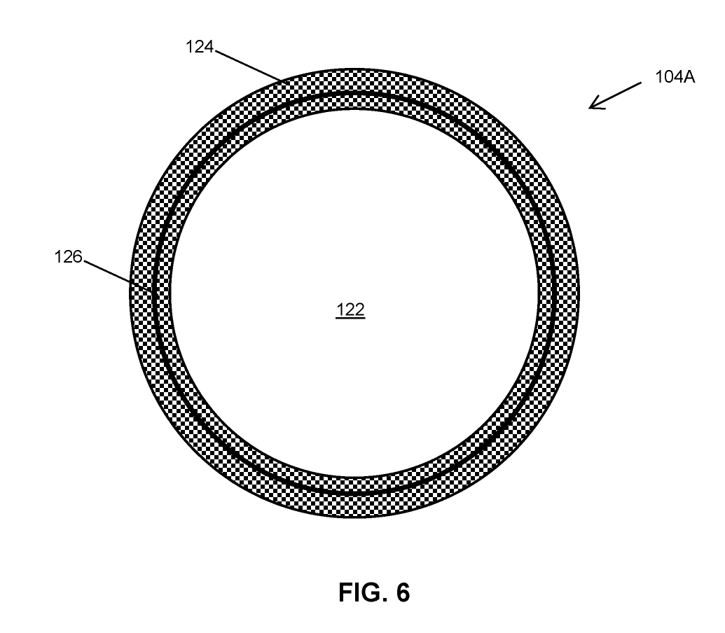

In one aspect, the first membrane and the second membrane comprise a constant membrane thickness selected to facilitate low-impedance transmission of ultrasound and thermal regulation as described above, as described above. In another aspect, illustrated in FIG. 6, the second membrane 104A may include a central membrane portion 122 and a reinforced peripheral portion 124. In this other aspect, the central membrane portion 122 is positioned and configured to facilitate low-impedance transmission of ultrasound and thermal regulation into the patient 18 (not illustrated) as described above. In this other aspect, the reinforced peripheral portion 124 may be reinforced by any suitable means including, but not limited to, locally increased membrane thickness, additional reinforcement elements attached to the second membrane 104 within the peripheral portion 124 such as a reinforcing layer, reinforcing strips, reinforcing fibers, and any other suitable additional reinforcement elements.

In this other aspect, the additional reinforcement elements may include textural elements to provide a no-slip membrane portion to enhance the immobilization of the patient 18 relative to the second membrane 104. Non-limiting examples of suitable textural elements include tacky or adhesive materials configured to adhere to an exposed surface 30 of the patient 18, a plurality of roughened or raised features distributed throughout the peripheral portion 124 to inhibit slipping of the second membrane 104 relative to the exposed surface 30 of the patient 18, and a circumferential ridge 126 to enable an enhanced seal enclosing the central membrane portion 122.

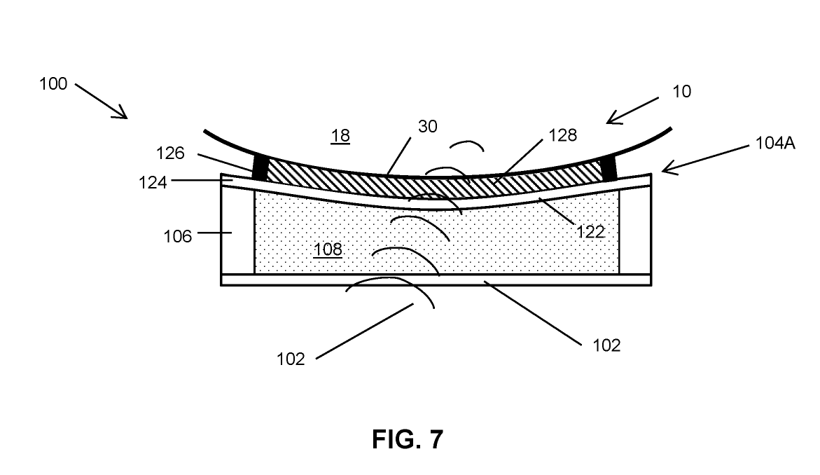

FIG. 7 is a cross-sectional view of the device 100 with the reinforced second membrane 104A as shown in FIG. 6 with an exposed surface 30 of the patient positioned in contact with the reinforced second membrane 104A. As illustrated in FIG. 7, the reinforced second membrane 104A is provided with a circumferential ridge 126 configured to seal against the exposed surface 30 of the patient 18 without impeding the transmission of HIFU 20 through the device 100 into the patient 18. In this aspect, the exposed surface 30 of the patient 18, the circumferential ridge 126, and the central membrane portion 122 of the second membrane 104A together enclose a sealed cavity 128. In one aspect, the sealed cavity 128 may be evacuated using a vacuum device as disclosed herein to enhance the intimate contact between the exposed outer surface 30 of the patient 18 and the central membrane portion 122 of the second membrane 104A. In another aspect, the sealed cavity 128 may be filled with an acoustic coupling gel to enhance the low impedance transmission of HIFU 20 into the patient 18. In this other aspect, a vacuum device may be used to remove any cavities or air pockets from the acoustic coupling gel within the sealed cavity 128 to further enhance the low-impedance transmission of HIFU 20 into the patient 18. In various additional aspects (not illustrated), the first membrane may be provided with similar reinforcement elements, textural elements, and sealing features configured to inhibit movement and enhance acoustic coupling of the device relative to the acoustic window of the MR-HIFU device.

In various aspects, the side wall of the device provides structural integrity to the device and further defines the first and second contours that shape the contours of the first and second membranes with the ultrasound window and the patient, respectively. The contours of the first and second membranes of the device, in particular the match of the contour of the first membrane to the ultrasound window of the MR-HIFU device and the match of the contour of the second membrane to the exposed surface of the patient influence the effectiveness of acoustic coupling at the respective interfaces. Because the side wall are not configured to transmit HIFU or exchange heat, the choice of materials from which to construct the side wall of the device are not constrained to be thermally or acoustically transmissive, as was the case with the materials of the first and second membranes. Any material with sufficient strength may be selected for construction of the continuous side wall without limitation.

In one aspect, the construction of the side wall of the device adjacent to the acoustic coupling fluid may be configured to inhibit the reflection of ultrasound waves. Without being limited to any particular theory, ultrasound waves reflected off of the side wall back into the acoustic coupling fluid may constructively or destructively interfere with ultrasound waves directed to the targeted deep tissue region and as a result degrade the effectiveness of the HIFU therapy. In one aspect, the side wall may be configured to transmit any impinging ultrasound waves to the surrounding air to reduce undesired reflection of ultrasound off of the sidewall into the acoustic coupling fluid. In another aspect, the material of the side wall may include materials configured to absorb ultrasound including, but not limited to, an ultrasound damping material such as a tungsten powder.

In another aspect, the side wall may be configured to form first and second contours of the first and second membranes that are custom-fitted to a particular acoustic window contour and individual patient's anatomy, respectively. In this other aspect, the side wall material may be a thermoplastic material characterized by flexibility at higher temperatures and rigidity at lower temperatures. In this aspect, the thermoplastic side wall may be heated to define the first and second contours of the first and second membranes in situ by coupling the device, the patient, and the ultrasound window of the MR-HIFU device and then allowing the side wall to cool and harden to retain the first and second contours so defined. In this aspect, a thermoplastic that softens at a relatively low temperature may be desired to avoid discomfort of the patient while defining the first and second contours in this manner. Non-limiting examples of thermoplastics suitable for use in the construction of the side wall include polyvinyl chloride (PVC), high-density polyethylene (HDPE), and polypropylene (PP).

In various aspects, the acoustic coupling fluid may be any suitable coupling fluid that is relatively matched to the acoustic impedance of the tissues of the patient to enhance the efficiency of transfer from the focused ultrasound transducer to the targeted deep tissues of the patient. Any known impedance-matched acoustic coupling fluid may be selected without limitation. Non-limiting examples of suitable acoustic coupling fluids include degassed and deionized water, oils such as mineral oil, and various ultrasound gels that may include propylene glycol, glycerin, phenoxyethanol, and/or or CARBAPOL® R 940 polymer.

In various aspects, the thermal management of the acoustic coupling fluid within the device is selected to provide sufficient heat transfer capacity without degrading or distorting the transmission of HIFU from the MR-HIFU device to the targeted tissue within the patient. In one aspect, the acoustic coupling fluid is relatively stationary within the device and a heat exchanger is provided within the closed interior volume in contact with the acoustic coupling fluid. In this one aspect, the device may include relatively high viscosity acoustic coupling fluid compositions, including but not limited to, various commercially available ultrasound gels. In another aspect, the device may include a recirculating heat exchanger device that recirculates and thermally conditions the acoustic coupling fluid from the closed internal volume of the device to enable heat exchange. In these other aspects, an acoustic coupling fluid composition with a lower viscosity including, but not limited to, degassed and deionized water, may be preferable.

In various aspects, recirculating acoustic coupling fluid may be constrained to flow through the closed interior volume of the device at a relatively low flow speed. Without being limited to any particular theory, high flow speeds of the acoustic coupling fluid are thought to interfere with the transmission of ultrasound through the moving medium. In addition, the presence of contaminants within the acoustic coupling fluid including, but not limited to, dissolved air bubbles, particulate matter, and/or ions may also reduce efficiency of ultrasound transmission. In one aspect, the acoustic coupling fluid may be conditioned during use to remove any of the contaminants described above. In another aspect, the flow conditions for the acoustic coupling fluid may be selected to inhibit the development of contaminants including, but not limited to, development of air bubbles due to cavitation.

In various aspects, the device may further include a fluid conditioning device operatively coupled to the inlet port and/or exit port outside of the closed interior volume. The fluid conditioning device may be configured to remove at least one contaminant from the acoustic coupling fluid received from the exit port, and to return the conditioned acoustic coupling fluid to the closed internal volume via the inlet port. Any suitable fluid conditioning device may be including without limitation including, but not limited to: filters, sieves, deionization devices, distillation devices, vacuum devices to remove gas bubbles, and any other suitable fluid conditioning device.

In various aspects, the device may further include one or more immobilization elements to immobilize the device with respect to the ultrasound window of the MR-HIFU system and/or to immobilize the patient with respect to the device. Without being limited to any particular theory, immobilization of the patient and device with respect to the MR-HIFU system may enhance the accuracy of targeting of the HIFU delivered to the targeting deep tissue region within the patient. In one aspect, the device may further include double-sided adhesive attached to a portion of the first membrane to reversibly secure the device to the ultrasound window of the MR-HIFU device. In another aspect, the device may further include a patient immobilization element operatively coupled to the continuous side wall. In this aspect, the patient securing device may be configured to immobilize the patient relative to the device. Non-limiting examples of suitable patient immobilization elements include one or more of a double-sided adhesive attached to a portion of the first membrane and ultrasound window, at least one pair of reversibly fastenable straps, and a vacuum immobilization device.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. From the above description and drawings, it will be understood by those of ordinary skill in the art that the particular embodiments shown and described are for purposes of illustrations only and are not intended to limit the scope of the present invention. References to details of particular embodiments are not intended to limit the scope of the invention.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A device to acoustically couple a patient to an MR-HIFU system, the device comprising:
a first membrane configured to acoustically couple to an ultrasound window of the MR-HIFU system;
a second membrane configured to acoustically couple to the patient at an exposed region overlying an ultrasound focus of the MR-HIFU system;
a continuous side wall comprising a first edge sealed to the first membrane and a second edge sealed to the second membrane; and
an acoustic coupling fluid contained within a closed internal volume defined by the first membrane, the second membrane, and the continuous side wall;
wherein the device is configured to form an impedance-matched ultrasound coupling between the ultrasound window and the exposed region of the patient.

2. The device according to claim 1, wherein the first edge further comprises a first contour, the first contour conforming to a window contour of the ultrasound window.

3. The device according to claim 2, wherein the second edge further comprises a second contour, the second contour conforming to an exposed region of the patient.

4. The device according to claim 3, wherein the continuous side wall is constructed of a thermoplastic material, wherein the continuous side wall is configured to deform when heated to form the first contour and the second contour, and is further configured to harden when cooled to maintain the first contour and the second contour during use.

5. The device according to claim 1, wherein the first membrane and the second membrane comprise an acoustically transmissive material with a membrane thickness of less than about 1 mm.

6. The device according to claim 5, wherein the acoustically transmissive material is selected from polyvylidene chloride and silicone.

7. The device according to claim 1, further comprising a heat exchange device operatively coupled to the acoustic coupling fluid, wherein the device is further configured to transfer heat between the exposed region of the patient and the acoustic coupling fluid via the second membrane.

8. The device according to claim 7, wherein the heat exchange device is a heat exchanger positioned within the closed internal volume.

9. The device according to claim 7, wherein:
the continuous side wall further defines an exit port configured to remove a portion of the acoustic coupling fluid from the closed internal volume and an inlet port configured to return the portion of the acoustic coupling fluid to the closed internal volume;
the heat exchange device is a recirculating heat exchange device operatively coupled to the exit port and the inlet port opposite to the closed internal volume; and
the recirculating heat exchange device is configured to exchange heat with the portion of the acoustic coupling fluid received via the exit port and to return the portion of the acoustic coupling fluid to the closed internal volume via the inlet port.

10. The device according to claim 9, further comprising a fluid conditioning device operatively coupled to at least one of the exit port and the inlet port, wherein the fluid conditioning device is configured to receive an amount of the acoustic coupling fluid containing at least one impurity, to remove the at least one impurity from the acoustic coupling fluid, and to return the acoustic coupling fluid to the closed internal volume via the inlet port, the at least one impurity selected from entrapped air, particulate matter, dissolved molecules, and any combination thereof.

11. The device according to claim 1, further comprising a double-sided adhesive attached to a portion of the first membrane opposite to the acoustic coupling fluid, the double-sided adhesive configured to reversibly secure the device to the ultrasound window of the MR-HIFU system.

12. The device according to claim 1, further comprising a patient immobilization element operatively coupled to the continuous side wall, the patient securing device configured to immobilize the patient relative to the second membrane of the device, wherein the patient immobilization element is selected from one or more of a double-sided adhesive attached to a portion of the second membrane opposite to the acoustic coupling fluid, at least one pair of reversibly fastenable straps, and a vacuum immobilization device.

13. A method of acoustically coupling a patient during treatment using an MR-HIFU system, the method comprising:
providing a device comprising:
a first membrane configured to acoustically couple to an ultrasound window of the MR-HIFU system;
a second membrane configured to acoustically couple to the patient at an exposed region overlying an ultrasound focus of the MR-HIFU system;
a continuous side wall comprising a first edge sealed to the first membrane and a second edge sealed to the second membrane; and
an acoustic coupling fluid contained within a closed internal volume defined by the first membrane, the second membrane, and the continuous side wall;
positioning the first membrane of the device adjacent to at least a portion of the ultrasound window of the MR-HIFU system; and
positioning the exposed region of the patient adjacent to the second membrane.

14. The method of claim 13, further comprising reversibly securing the device to the ultrasound window of the MR-HIFU system using a double-sided adhesive attached to a portion of the first membrane opposite to the acoustic coupling fluid.

15. The method of claim 14, further comprising providing a patient immobilization element operatively coupled to the continuous side wall, and immobilizing the patient relative to the device using the patient immobilization element.

16. The method of claim 13, further comprising:
providing a heat exchange device operatively coupled to the acoustic coupling fluid of the device; and
modulating a temperature of the exposed region of the patient by transferring heat between the exposed region of the patient and the acoustic coupling fluid, and modulating a fluid temperature of the acoustic coupling fluid using the heat exchange device.

17. The method of claim 13, further comprising:
providing a fluid conditioning device operatively coupled to the closed internal volume of the device;
receiving an amount of the acoustic coupling fluid containing at least one impurity from the closed internal volume;
removing the at least one impurity from the acoustic coupling fluid; and
returning the amount of the acoustic coupling fluid to the closed internal volume;
wherein the at least one impurity selected from entrapped air, particulate matter, dissolved molecules, and any combination thereof.

18. A system for administering an MR-HIFU treatment to a patient, comprising:
an MR-HIFU system comprising an ultrasound window and an ultrasound transducer configured to deliver HIFU to an ultrasound focus; and
a device comprising:
a first membrane configured to acoustically couple to the ultrasound window;
a second membrane configured to acoustically couple to the patient at an exposed region overlying the ultrasound focus;
a continuous side wall comprising a first edge sealed to the first membrane and a second edge sealed to the second membrane; and
an acoustic coupling fluid contained within a closed internal volume defined by the first membrane, the second membrane, and the continuous side wall;
wherein the device is configured to form an impedance-matched ultrasound coupling between the ultrasound window and the exposed region of the patient.

19. The system of claim 18, further comprising a heat exchange device operatively coupled to the acoustic coupling fluid of the device, wherein the device is further configured to transfer heat between the patient and the acoustic coupling fluid via the second membrane and the heat exchange device is configured to regulate a temperature of the acoustic coupling fluid.

20. The system of claim 19, further comprising a fluid conditioning device operatively coupled to the closed internal volume, wherein the fluid conditioning device is configured to receive an amount of the acoustic coupling fluid containing at least one impurity from the closed internal volume, to remove the at least one impurity from the acoustic coupling fluid, and to return the acoustic coupling fluid to the closed internal volume, the at least one impurity selected from entrapped air, particulate matter, dissolved molecules, and any combination thereof.
